(19) United States
(12) Patent Application Publication
(10) Pub. No.: US 2023/0227457 A1
(43) Pub. Date: Jul. 20, 2023

(54) IMPROVED PROCESS FOR PREPARATION OF SITAGLIPTIN

(71) Applicant: AARTI INDUSTRIES LIMITED, Mumbai (IN)

(72) Inventors: Parimal Hasmukhlal DESAI, Mumbai (IN); Narendra Jagannath SALVI, Mumbai (IN); Bharatkumar Surendra PATRAVALE, Mumbai (IN); Chetan Liladhar SALUNKE, Mumbai (IN); Rajendra Babasaheb YADAV, Mumbai (IN); Navnath Gorakshanath SALPURE, Mumbai (IN); Nitin Baburao KAJALE, Mumbai (IN)

(21) Appl. No.: 18/001,222

(22) PCT Filed: Jun. 11, 2021
(86) PCT No.: PCT/IN2021/050567

§ 371 Date: Dec. 8, 2022

(30) Foreign Application Priority Data
Jun. 12, 2020 (IN) 202021024760

(51) Int. Cl. C07D 487/04

(52) U.S. Cl. CPC C07D 487/04

(57) ABSTRACT

A process for the preparation of specific enantiomeric Sitagliptin with good chiral purity and higher yield using improved biocatalyst and by engineering an enzyme to mediate the efficient conversion of ketoamide to obtain enatiomerically pure Sitagliptin in presence of an amino group donor.

### FIELD OF THE INVENTION

The present invention relates to an improved process for the preparation of a dipeptidyl peptidase-4 (DPP-4) inhibitor and in particular preparation of desired stereoisomer of Sitagliptin.

### BACKGROUND AND PRIOR ART

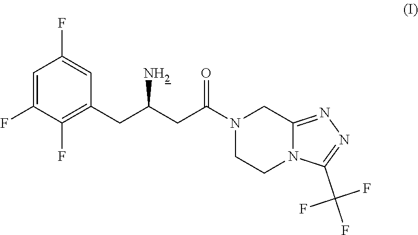 [SMILES: [NH-][C@@H](CC(=O)N1CCn2c(nnc2C(F)(F)F)C1)Cc1cc(F)cc(F)c1F]

Sitagliptin of Formula (I) is developed and marketed as the phosphate salt under the trade name Januvia by Merck and Co. It is an oral anti-diabetic drug of dipeptidyl peptidase-4 (DPP-4) inhibitor class, chemically known as (R)-4-oxo-4-[3-(trifluoromethyl)-5,6-dihydro[1,2,4]triazolo[4,3-a]pyrazin-7(8H)-yl]-1-(2, 4,5-trifluorophenyl)butan-2-amine. Sitagliptin is used to treat high blood sugar level caused by type 2 diabetes and also used to avoid smoking. Incretin hormone regulates the production and release of insulin. Sitagliptin works by protecting incretin hormones, so they aren't broken down too quickly. This helps the body to use insulin better way and lowers your blood sugar. Sitagliptin is used along with lifestyle changes such as improved diet and exercise.

Sitagliptin is first claimed in U.S. Pat. No. 6,699,871 and Sitagliptin dihydrogen phosphate salt or its hydrate is specifically claimed in U.S. Pat. No. 7,326,708. The process disclosed in U.S. Pat. No. 6,699,871 for preparation of Sitagliptin, involves coupling of (3R)-3-[1,1-dimethylethoxycarbonylamino]-4-(2,4,5)-trifluorophenyl)-butanoic acid with 3-(trifluoromethyl)-5,6,7,8-tetrahydro-1,2,4-triazolo [4,3-a]-pyrazine in presence of HOBt and EDC in MDC.

(3R)-3-[1,1-dimethylethoxycarbonylamino]-4-(2,4,5)-trifluorophenyl)-butanoic acid is prepared by reacting (2S)-2,5-dihydro-3,6-dimethoxy-2-isopropyl-pyrazine with 2,4,5-trifluoromethyl benzyl bromide in presence of butyl lithium followed by reaction with Di-BOC. Various processes for synthesis of Sitagliptin and its pharmaceutically acceptable salts are known.

WO2019158285 and WO2006081151 discloses amination of ketoamide to form enamine using ammonium acetate. Further enamine is converted to Sitagliptin using catalyst such as bis((1,5-cyclooctadiene)(chloro)rhodium) and ligand such as Josiphos SL-J 002-1. During resolution step using chemical procedures, theoretically 50-60% of the total material can be isolated as a pure enantiomer. Wastage of unwanted material makes the process costly and recycling of the wrong isomer requires extra unit operations and cost.

WO2004085661 discloses conversion of ketoamide to Sitagliptin using (S)-phenylglycine amide (S-PGA) as a chiral auxillary to form Z-enamine. Phenylglycine protected enamine obtained is hydrogenated in presence of platinum oxide. Phenylglycine protected Sitagliptin is deprotected using palladium oxide to yield Sitagliptin. However use of metal catalyst leave trace amount of metal in the final product, which is problematic for manufacture of pharmaceutical products. This may need additional purifications which ultimately results in yield loss. Therefore, chemical processes comparatively are not efficient to prepare Sitagliptin at low cost as they consume more solvents and chemicals, which are difficult to handle at large scale and are not environment friendly.

Enzymes are known to have unique stereoselective property of producing specific enantiomer with desired chiral purity. Further, the enzymes can be recovered and recycled after the completion of a process and hence, reduces the cost of producing products. Enzymatic process of preparing enantiomerically pure Sitagliptin is known. 2805/MUM/2010 discloses such a process for preparation of hydroxyl compound of Formula (I) from keto compound using oxidoreductase or ketoreductase that selectively reduces keto to hydroxy in presence of a suitable co-factor. The enzyme/polypeptide used in cited patent is of “Ketoreductase” type, which is capable of stereoselectively reducing a ketone to a hydroxy compound. The hydroxy compound obtained can be converted to Sitagliptin in three steps including a) Mesylation of the hydroxy compound, b) Nucleophilic substitution of mesyl intermediate to get azide intermediate and c) Conversion of azide intermediate to amine (Sitagliptin). However, the three stage conversion also causes loss of yields during intermediate conversions.

Thus there is a need for process for improving the percentage conversion and stereoselectivity under mild conditions using enzymes having unique stereoselective property of producing specific enantiomeric Sitagliptin with good chiral purity and higher yield and by engineering the enzyme to mediate the efficient conversion of ketoamide to obtain enantiomerically pure Sitagliptin in presence of an amino group donor.

### SUMMARY OF THE INVENTION

In a general aspect, the present invention provides a process of preparing Sitagliptin of Formula (I)

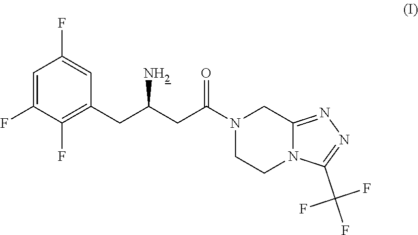 [SMILES: [NH-][C@@H](CC(=O)N1CCn2c(nnc2C(F)(F)F)C1)Cc1cc(F)cc(F)c1F]

The process comprises reacting a ketoamide of Formula (II)

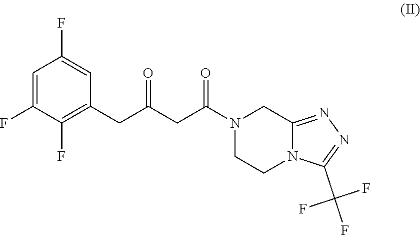 [SMILES: O=C(CC(=O)N1CCn2c(nnc2C(F)(F)F)C1)Cc1cc(F)cc(F)c1F]

with an amino compound of Formula (III)

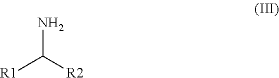

where R1 and R2 is selected from alkyl, alkylaryl or aryl

in a buffer at a pH 8 to 9 in presence of a biocatalyst pyridoxal-5-phosphate (PLP) and a transaminase enzyme CDX-036 at a temperature of 35° C. to 60° C.

In an embodiment, the process comprises adding a co-solvent selected from dimethyl sulfoxide (DMSO), dimethyl formamide, methyl tert-butyl ether (MTBE), isopropyl acetate, methanol, ethanol, isopropanol, or water maintaining the temperature of the mixture to 35° C. to 60° C.

In an embodiment, the process comprises preparing a solution of ketoamide of Formula (II) in a co-solvent selected from dimethyl sulfoxide (DMSO), ethyl acetate, chloroform, methylene dichloride (MDC), acetone, methanol, ethanol, isopropanol.

The process comprises preparing a mixture of amino compound solution, and a solvent selected from water in a buffer and adding the biocatalyst at a pH of 8 to 9.

The amino compound is selected from isopropylamine, alanine, ortho-xylylenediamine, 1-phenylethylamine, 3-aminocyclohexa-1,5-dienecarboxylic acid, 1,2-diaminoethane, 1,4-diaminobutane and 1,5-diaminopentane. The buffer is triethanolamine and the biocatalyst is pyridoxal-5-phosphate (PLP).

In an aspect, the process of reacting ketoamide with the amino compound optionally comprises adding a surface tension reducing agent or a phase transfer catalyst.

The process comprises adding the surface tension reducing agent or the phase transfer catalyst to the solution of amino compound before adding the biocatalyst. The process of reacting ketoamide with the amino compound is at a temperature of 35-60° C.

The surface tension reducing agent is selected from didecyldimethylammonium chloride (DDAC), cetyltrimethylammonium chloride (CTAC), or cetyltrimethylammonium bromide (CTAB) and the phase transfer catalyst is selected from tetrabutylammonium bromide (TBAB), tetrabutylammonium fluoride (TBAF), tetrabutylammonium hydroxide (TBAH), or triethylbenzylammonium chloride (TEBA).

In embodiment, the chiral purity of the Sitagliptin is 99.90% to 99.99%.

In another aspect, the present invention relates to a process for preparing salt of Sitagliptin. The process comprises reacting Sitagliptin with an acid in presence of a solvent and heating to a reflux temperature. The acid is selected from conc. HCl, orthophosphoric acid, maleic acid, fumaric acid.

In an embodiment, the salt of Sitagliptin is an acid salt or a hydrate or a solvate.

### DETAILED DESCRIPTION OF THE INVENTION

In an embodiment, the present invention relates to an improved process for preparation of Sitagliptin of Formula (I).

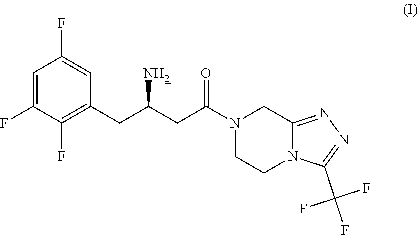 [SMILES: [NH-][C@@H](CC(=O)N1CCn2c(nnc2C(F)(F)F)C1)Cc1cc(F)cc(F)c1F]

The process comprises reacting a ketoamide of Formula (II)

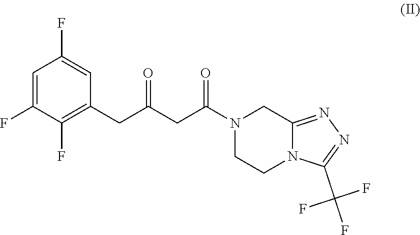 [SMILES: O=C(CC(=O)N1CCn2c(nnc2C(F)(F)F)C1)Cc1cc(F)cc(F)c1F]

with an amino compound of Formula III

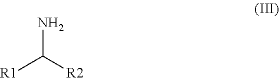

where R1 and R2 is selected from alkyl, alkylaryl or aryl in buffer, at a pH 8 to 9 in presence of a biocatalyst pyridoxal-5-phosphate (PLP) and transaminase enzyme CDX-036.

In a transaminase reaction the compound of Formula (III) donates the amino group to the amino group acceptor i.e. ketoamide of Formula (II) forming the compound of Formula (I).

In an embodiment, a solution of Ketoamide of Formula (II) can be prepared in a co-solvent. The co-solvent can be selected from dimethyl sulfoxide (DMSO), ethyl acetate, chloroform, methylene dichloride (MDC), acetone, methanol, ethanol, or isopropanol and preferably carried out in DMSO. Further, a mixture of amino compound (III) solution, a solvent selected from water and a buffer selected from triethanolamine is prepared and pH is adjusted 8 to 9.

In an embodiment, the amino compound can be a compound which is capable of donating amino group to the amino group acceptor i.e. ketoamide of Formula (II). The amino compound can be selected from isopropylamine, alanine, ortho-xylenediamine, 1-phenylethylamine, 3-aminocyclohexa-1,5-dienecarboxylic acid, 1,2-diaminoethane, 1,4-diaminobutane and 1,5-diaminopentane. Preferably, the amino compound can be isopropylamine. The amino compound and water along with the buffer forms a buffer system. To said buffer system, the biocatalyst can be added. The biocatalyst can be selected from pyridoxal-5-phosphate (PLP). Preferably, the biocatalyst can be pyridoxal-5-phosphate (PLP) obtained by conventionally known chemical process and acts as coenzyme. To the ketoamide (Formula II) solution, the amino compound in buffer system containing the biocatalyst is added forming the reaction mixture. The pH of the reaction mixture can be maintained at pH 8 to 9. To this reaction mixture, the transaminase enzyme CDX-036 can be added. Transaminase, also called aminotransferase, is a type of enzyme that catalyzes the reversible transfer of amino groups between amino compounds and carbonyl compounds. The transaminase enzyme can be preferably recombinant or genetically engineered transaminase enzyme and can be obtained from United States (Company name: Codexis, Inc.) as “CDX-036”. The Transaminase enzyme aids a stereospecific conversion of the ketoamide directly to an amino compound i.e. Sitagliptin thereby reducing multiple steps in the conversion as in known methods. The conversion of ketoamide to Sitagliptin by transamination reaction aided by the enzyme also contributes to high yield of Sitagliptin.

The addition of transaminase enzyme can be carried out in presence of a co-solvent selected from dimethyl sulfoxide (DMSO), dimethyl formamide, ethers like MTBE and esters like isopropyl acetate, methanol, ethanol, isopropanol, water and mixtures thereof and preferably carried out in presence of DMSO. The reaction mixture can be heated to a temperature of 35° C. to 60° C. and maintaining pH of the mixture between 8 to 9. At this stage, enzyme can be gradually added to the co-solvent over period of time maintaining a temperature of the mixture to 35° C. to 60° C., preferably at 40° C.-55° C. and more preferably at 51-52° C. till completion of the reaction. The amino group of compound of Formula (III) can be transferred to the coenzyme to produce a carbonyl byproduct while the biocatalyst such as pyridoxal-5′-phosphate can be converted to pyridoxamine phosphate. The transfer of the amino group from pyridoxamine phosphate to the compound of Formula (II) produces a chiral amine and regenerates the coenzyme. The reaction can be monitored and after completion of the reaction pH was adjusted to 2 to 3 using an acid selected from concentrated hydrochloric acid, sulfuric acid, nitric acid, acetic acid and orthophosphoric acid. The reaction mass can be stirred for 2 hours and filtered extracting the Sitagliptin base.

In an embodiment, the process optionally comprises adding a surface tension reducing agent (surfactant) or a phase transfer catalyst. The surface tension reducing agents or the surfactants can be selected from didecyldimethylammonium chloride (DDAC), Cetyltrimethylammonium chloride (CTAC) (Cetyltrimethylammonium bromide CTAB, cetrimide). The phase transfer catalyst can be selected from tetrabutylammonium bromide (TBAB), tetrabutylammonium fluoride (TBAF), tetrabutylammonium hydroxide (TBAH), Triethylbenzylammonium chloride (TEBA). The surfactants or phase transfer catalyst can be preferably added to the buffer system comprising amino compound of Formula (III) before adding the biocatalyst Pyridoxal-5-phosphate. It is found that use of phase transfer catalyst or surfactants in the process improves conversion and consequently good yield.

It was observed that ketoamide of Formula (II) is converted to an imine compound due to acid treatment during work up. As the imine formation increases, the conversion and yield decreases. This also reduces the reaction rate, which can be clearly seen from the TABLE 1 below:

TABLE 1
Reaction rate/conversion rate comparison of Sitagliptin
base formation from pro-sitagliptin ketone (Ketoamide)
with and without use of Co-Solvent DMSO or surface
tension reducing agent (surfactant) CTAC

| Co-Solvent/ | | % Conversion |
|---|---|---|
| Surfactant | Reaction conditions/Remark | (Reaction mass) |
| DMSO 3 V | pH = 8.4 to 8.6; Temp = 45° C. | SITA = 74.86 |
| | Under N₂ flow | Ketoamide = 18.34 |
| | Remark: Ketoamide added as | Imine = 3.49 |
| | solution in 2 V DMSO over 3 hrs | |
| | at 45° C. | |
| CTAC 30% | pH = 8.5; Temp = 45° C. | SITA = 81.81 |
| aqueous | Under N₂ flow | Ketoamide = 2.49 |
| solution, 15 V | Remark: Ketoamide added as | Imine = 1.57 |
| | solid at start of reaction. | |
| No co solvent | pH = 8.4 to 8.7; Temp = 45° C. | SITA = 43.45 |
| or surfactant | Under N₂ flow | Ketoamide = 26.81 |
| used. | Remark: Ketoamide added as | Imine = 26.03 |
| | solid over 25 hrs. | |

The process of preparing the compound of Formula (I) further comprises extracting the compound of Formula (I) by washing the filtrate with ethyl acetate adjusting the pH of the aqueous layer to 11 to 12, extracting the mass with ethyl acetate or isopropyl acetate solvent, washing the organic layer with brine and drying over sodium sulfate and distilling the solvent to obtain Sitagliptin base. The pH of the reaction mass can be adjusted to pH 2.0 to pH 3.0 using acid. The reaction mass can then be stirred at 35° C. to 60° C. forming Sitagliptin of Formula (I).

In an embodiment, the chiral purity of the Sitagliptin base prepared by the process of present invention is not less than 99.90% (99.90% to 99.99%).

In another embodiment, the present invention provides a process of preparing a salt of Sitagliptin. The process comprises reacting Sitagliptin with a concentrate acid selected from hydrochloric acid, orthophosphoric acid, malic acid, fumaric acid in presence of a solvent selected from isopropyl alcohol (IPA), methyl ethyl ketone, ethanol, or methanol and heating to a temperature of 75° C. to 70° C. The salt can be selected from an acid salt, a hydrate or a solvate salt of Sitagliptin. The Sitagliptin salt prepared by the process of the present invention can have a chiral purity of not less than 99.90% (99.90% to 99.99%).

### EXAMPLES

Example and implementation is provided herein below for illustration of the invention. Variations, modifications, and enhancements to the described examples and implementations and other implementations can be made based on what is disclosed.

### Example 1

### Preparation of Sitagliptin

Water (140 ml) and triethanolamine (8.45 ml) was charged to 4M isopropylamine solution (133 ml) at room temperature. The pH of the mixture was adjusted to 8.5 using conc. HCl (about 55 ml). To this buffer system Pyridoxal 5-phosphate (335 mg) was added and reaction mixture was stirred for 15-20 minutes. Engineered transaminase enzyme “CDX-036” (2 gm) was added and the reaction mixture was stirred for 30 minutes. To this reaction mass, DMSO (111 ml) was added gradually. The reaction mass was heated to 45° C. and temperature was maintained till end of reaction. Separately prepared solution of 1-[3-(trifluoromethyl)-6,8-dihydro-5H-[1,2,4]triazolo[4,3-a]pyrazin-7-yl]-4-(2,3,5-trifluorophenyl)butane-1,3-dione (Ketoamide) of Formula (II) (50 gm) in DMSO (55 ml) was charged to the above reaction mixture under inert atmosphere. The pH of the mixture was maintained to 8.4 to 8.7 throughout the reaction by gradual addition of 4M isopropylamine solution. After completion of reaction (after 36 hrs), the pH of reaction mass was adjusted to 2.0-3.0 using conc. HCl. The reaction mass was stirred at 45° C. for 2 hours and filtered. The filtrate was washed with ethyl acetate (250 ml×3 times) and pH of aqueous layer was adjusted to 11.0-11.5 using 50% sodium hydroxide solution. The reaction mass was extracted with ethyl acetate (250 ml×3 times). The organic layer was then washed with brine (250 ml), dried over sodium sulfate and distilled to get Sitagliptin base (34.2 gm) (68% yield). HPLC purity: 99.57%; Chiral purity: 99.92%.

### Example 2

### Preparation of Sitagliptin (with Surface Tension Reducing Agent)

The solution of 1-[3-(trifluoromethyl)-6,8-dihydro-5H-[1,2,4]triazolo [4,3-a]pyrazin-7-yl]-4-(2,3,5-trifluorophenyl)butane-1,3-dione (Ketoamide) of Formula (II) was prepared by dissolving (50 gm) in DMSO (55 ml). Water (100 ml) and triethanolamine (8.5 ml) was charged to 4M isopropylamine solution (130 ml) and the mixture was stirred. Cetyltrimethylammonium chloride (CTAC) (30% aqueous solution) (50 ml) was added to the above mixture at room temperature. The pH of this mixture was adjusted to 8.5 using conc. HCl. Pyridoxal-5-phosphate (335 mg) was added to this mixture and reaction mass was stirred for 15-20 minutes. To this mixture the above ketoamide solution was charged. Engineered transaminase enzyme “CDX-036” (2 gm) was added and reaction mixture was stirred for 30 minutes. To this reaction mixture, DMSO (95 ml) was added gradually. The reaction mixture was heated to 45° C. and maintained at this temperature till end of reaction. The reaction assembly was provided with arrangements for pH monitoring, Nitrogen purging and addition of 4M isopropylamine solution (for pH maintaining). The pH of the mixture was adjusted to 8.4-8.7 and the ketoamide solution was then added to reaction mass over a period of 3 hours in an inert atmosphere. pH of the mixture was continuously monitored and maintained between 8.4 and 8.7. After completion of reaction (38 hrs), the pH of reaction mass was adjusted to 2.0-3.0 using conc. HCl. The reaction mass was stirred at 45° C. for 2 hours and filtered. The filtrate was washed with ethyl acetate (250 ml×3 times) and pH of aqueous layer was adjusted to 11.0-11.5 using 50% sodium hydroxide solution. The reaction mass was then extracted with isopropyl acetate (250 ml×3 times). The organic layer was then washed with brine (250 ml), dried over sodium sulfate and distilled to get 42 gm (84% yield) of Sitagliptin base. HPLC purity: 97.11%; Chiral purity: 99.96%.

### Example 3

### Preparation of Sitagliptin

4M isopropylamine solution (164 ml) was charged to the mixture of Water (192 ml) and triethanolamine (10.86 ml) at 25-30° C. The mixture was stirred well and cooled to 20-25° C. The pH of the mixture was adjusted to 8.5 using conc. HCl. Buffer system 670 mg was charged to the reaction mixture at 20-25° C. The pH was adjusted to 8.5 using 4M isopropylamine. Engineered transaminase enzyme “CDX-036” (5 gm) and DMSO (110 ml) was added with the continuous stirring. The pH is adjusted to 8.5 using 4M isopropylamine. The reaction mixture was heated to 50 to 52° C. The solution of 1-[3-(trifluoromethyl)-6,8-dihydro-5H-[1,2,4]triazolo[4,3-a]pyrazin-7-yl]-4-(2,3,5-trifluorophenyl)butane-1,3-dione (Ketoamide) of Formula (II) (100 gm) in DMSO (110 ml) was prepared separately and lotwise to the reaction mixture. First lot of the ketoamide solution (105 gm) was charged gradually to the reaction mixture under inert atmosphere at 50-52° C. under continuous stirring in 1.5-2 hours. The pH of the mixture was maintained to 8.5 throughout the reaction by gradual addition of 4M isopropylamine solution. Second lot of water (192 ml) and triethanolamine (6 ml) was charged to the reaction mass at 50-52° C. Isopropylamine (24 gm) was charged to the reaction mixture. Second lot of Ketoamide solution (105 gm) was added gradually in 70-90 minutes at 50-52° C. The pH of the reaction mixture was adjusted to 8.4 to 8.6 using 50% HCl solution at 45° C. The reaction was maintained for 2-3 hours at 50-55° C. The mass was cooled to 30-35° C. and MDC (500 ml) was added. The mass was stirred well and filtered. The filtrate was extracted with MDC (500 ml). The layers were separated and aqueous layer was washed with MDC (500 ml). All the MDC layers were mixed and dilute HCl (200 ml) was charged and stirred for 0.5 hours. The layers were settled and separated. To the MDC layer dilute HCl (200 ml) was charged and stirred for 0.5 hours. The layers were separated and MDC layer was set aside for recycling of ketoamide. To the aqueous layer, MDC (300 ml) was charged and the pH was adjusted to 11.5 using 40% sodium hydroxide solution. The layers were separated aqueous layer was washed with MDC (500 ml×2). MDC layer was distilled under vacuum to yield Sitagliptin (59.4 gm). Yield: 59.4 gm (Yield: 60%), HPLC purity: 99.9%, Chiral purity: 99.97%.

### Example 4

### Recovery of 1-[3-(trifluoromethyl)-6,8-dihydro-5H-[1,2,4]triazolo[4,3-a]pyrazin-7-yl]-4-(2,3,5-trifluorophenyl)butane-1,3-dione

The MDC layer set aside for the recovery from example 3 is distilled off to yield oily residue. Acetonitrile (40 ml) was charged to the above oil and heated at 45-50° C. Water (150 ml) was added gradually to the clear solution at 45-50° C. The mass was cooled gradually to RT and then to 10-15° C. The slurry obtained was filtered and washed with water (40 ml×2). The 1-[3-(trifluoromethyl)-6,8-dihydro-5H-[1,2,4]triazolo[4,3-a]pyrazin-7-yl]-4-(2,3,5-trifluorophenyl)butane-1,3-dione obtained was dried at 50° C. for 9-10 hours. Dry weight: 28 gm (Yield: 70%). HPLC purity: 99.69%.

The ketoamide 1-[3-(trifluoromethyl)-6,8-dihydro-5H-[1,2,4]triazolo[4,3-a]pyrazin-7-yl]-4-(2,3,5-trifluorophenyl)butane-1,3-dione recovered was recycled back for preparation of Sitagliptin.

### Example 5

### Preparation of Sitagliptin from Recovered 1-[3-(trifluoromethyl)-6,8-dihydro-5H-[1,2,4]triazolo[4,3-a]pyrazin-7-yl]-4-(2,3,5-trifluorophenyl)butane-1,3-dione from Example 4

4M isopropylamine solution (41 ml) was charged to the mixture of Water (48 ml) and triethanolamine (2.71 ml) at 25-30° C. The mixture was stirred well and cooled to 20-25° C. The pH of the mixture was adjusted to 8.5 using conc. HCl. PLP (670 mg) was charged to the reaction mixture at 20-25° C. The pH was adjusted to 8.5. Engineered transaminase enzyme “CDX-036” (1.25 gm) and DMSO (27.5 ml) was added with the continuous stirring. The pH is adjusted to 8.5 using 4M isopropylamine. The reaction mixture was heated to 50-52° C. The solution of 1-[3-(trifluoromethyl)-6,8-dihydro-5H-[1,2,4]triazolo[4,3-a]pyrazin-7-yl]-4-(2,3,5-trifluorophenyl)butane-1,3-dione (Ketoamide) obtained in example 4 (25 gm) in DMSO (27.5 ml) was prepared separately and lotwise to the reaction mixture. First lot of the ketoamide solution (25 gm) was charged gradually to the reaction mixture under inert atmosphere at 50-52° C. under continuous stirring in 1.5-2 hours. The pH of the mixture was maintained to 8.5 throughout the reaction by gradual addition of 4M isopropylamine solution. Second lot of water (48 ml) and triethanolamine (1.5 ml) was charged to the reaction mass at 50-52° C. Isopropylamine (6 gm) was charged to the reaction mixture. Second lot of Ketoamide solution (25 gm) was added gradually in 70-90 minutes at 50-52° C. The pH of the reaction mixture was adjusted to 8.4 to 8.6 using 50% HCl solution at 45° C. The reaction was maintained for 2-3 hours at 50-52° C. The mass was cooled to 30-35° C. and MDC (125 ml) was added. The mass was stirred well and filtered. The layers were separated. The aqueous layer was extracted with MDC (2×100 ml). The pH of the combined MDC layer was adjusted to less than 2 using dilute HCl. The layers were separated. MDC (125 ml) was charged to the combined aqueous layers and the pH was adjusted to 11.5 using 40% sodium hydroxide solution. The layers were separated and aqueous layer was washed with MDC (2×125 ml). The organic layer was dried over sodium sulfate and MDC layer was distilled under vacuum to yield Sitagliptin (16 gm) (Yield: 64%). HPLC purity: 99.9%, Chiral purity 99.95%.

### Example 6

### Preparation of Sitagliptin Hydrochloride

IPA (10 ml) was added to Sitagliptin base (1.2 gm) obtained in Example 1 or 2. The mixture was heated to 75-70° C. and conc. HCl (0.4 ml) was slowly added to the mixture. The reaction mass was then cooled to room temperature and stirred at this temperature for 1 hour. The precipitated solid was the filtered and dried to get 1.2 gm of Sitagliptin Hydrochloride. HPLC purity: 99.8%; Chiral purity: 99.99%.

### Example 7

### Synthesis of Sitagliptin Phosphate

IPA (20 ml) was charged to the Sitagliptin base (10 gm) and the mixture was heated to 70-75° C. Orthophosphoric acid (2.6 ml) and water (9 ml) was slowly charged to the mixture. The reaction mass was stirred at 70-75° C. for 1 hour and cooled to 50-55° C. IPA (37.5 ml) was the added to reaction mass at 50-55° C. and stirred at this temperature for 3 hours. The mixture was cooled to 25-30° C. and stirred for 1 hour. The reaction mass was further cooled to 5-10° C. and stirred for 1 hour. The reaction mass was filtered and dried to get Sitagliptin Phosphate (10.2 gm). Purity: 99.8%; Chiral purity: 99.9%.

### Example 8

### Preparation of Sitagliptin Phosphate Monohydrate

Sitagliptin (16.0 g) obtained in any of the above examples was dissolved in IPA (34 mL) and H₂O (14.4 mL), and 85% phosphoric acid H₃PO₄ (4.54 g) was added dropwise. The mixture was heated to 75° C. to dissolve the solids. The batch was then cooled to 60-65° C. The mass was maintained for 1 h and cooled to ambient temperature. IPA (56 mL) was added, and the batch was stirred for 1 h. The batch was filtered, and the wet cake was washed with IPA (5 mL). The wet cake was dried at 60° C. to give 9.83 g of sitagliptin phosphate monohydrate (95.0%) with a purity of 99.93%.

The foregoing description of specific embodiments of the present invention has been presented for purposes of illustration and description. They are not intended to be exhaustive or to limit the present invention to the precise forms disclosed, and obviously many modifications and variations are possible in light of the above teaching.

The embodiments were chosen and described in order to best explain the principles of the present invention and its practical application, to thereby enable others, skilled in the art to best utilize the present invention and various embodiments with various modifications as are suited to the particular use contemplated.

It is understood that various omission and substitutions of equivalents are contemplated as circumstance may suggest or render expedient, but such are intended to cover the application or implementation without departing from the spirit or scope of the present invention.

1. A process of preparing Sitagliptin of Formula (I) 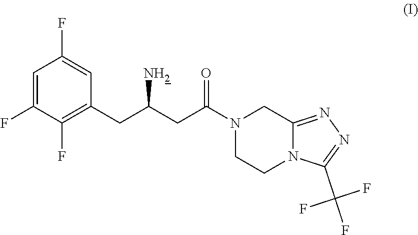 [SMILES: [NH-][C@@H](CC(=O)N1CCn2c(nnc2C(F)(F)F)C1)Cc1cc(F)cc(F)c1F]
comprising:
reacting a ketoamide of Formula (II) 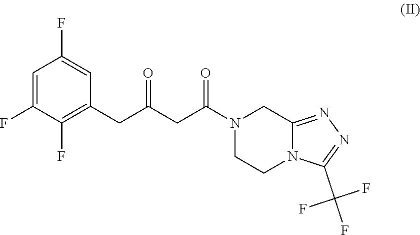 [SMILES: O=C(CC(=O)N1CCn2c(nnc2C(F)(F)F)C1)Cc1cc(F)cc(F)c1F]
with an amino compound of Formula (III) 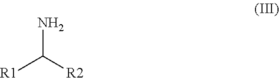
where R1 and R2 is selected from alkyl, alkylaryl or aryl in a buffer in presence of a biocatalyst pyridoxal-5-phosphate (PLP) and a transaminase enzyme CDX-036.

2. The process as claimed in claim 1, further comprises adding a co-solvent selected from dimethyl sulfoxide (DMSO), dimethyl formamide, methyl tert-butyl ether (MTBE), isopropyl acetate, methanol, ethanol, isopropanol, and water, and maintaining the temperature in a range from 35° C. to 60° C.

3. The process as claimed in claim 1, further comprises preparing a solution of ketoamide of Formula (II) in a co-solvent selected from dimethyl sulfoxide (DMSO), ethyl acetate, chloroform, methylene dichloride (MDC), acetone, methanol, ethanol, and isopropanol.

4. The process as claimed in claim 1, further comprises preparing p mixture of the amino compound of Formula (II) solution, and a solvent selected from water in a buffer, and adding the biocatalyst at a pH of 8 to 9.

5. The process as claimed in claim 1, wherein the amino compound of Formula (III) is selected from isopropylamine, alanine, ortho-xylenediamine, 1-phenylethylamine, 3-aminocyclohexa-1,5-dienecarboxylic acid, 1,2-diaminoethane, 1,4-diaminobutane, and 1,5-diaminopentane.

6. The process as claimed in claim 1, wherein the buffer is triethanolamine.

7. The process as claimed in claim 1, wherein reacting the ketoamide of Formula (II) with the amino compound of Formula (III) optionally comprises adding a surface tension reducing agent or a phase transfer catalyst.

8. The process as claimed in claim 7, further comprises adding the surface tension reducing agent or the phase transfer catalyst to a solution of amino compound before adding the biocatalyst.

9. The process as claimed in claim 8, wherein the surface tension reducing agent is selected from didecyldimethylammonium chloride (DDAC), cetyltrimethylammonium chloride (CTAC), and cetyltrimethylammonium bromide (CTAB).

10. The process as claimed in claim 1, wherein reacting the ketoamide of Formula (II) with the amino compound of Formula (III) is at a temperature of 35-60° C.

11. The process as claimed in claim 1, wherein the chiral purity of the Sitagliptin ranges from 99.90% to 99.99%.

12. A process for preparing a salt of Sitagliptin comprises reacting Sitagliptin as claimed in claim 1 in presence of a solvent and heating to a reflux temperature with an acid.

13. The process as claimed in claim 12, wherein the salt of Sitagliptin is an acid salt, a hydrate, or a solvate.

14. The process as claimed in claim 1, wherein reacting the ketoamide of Formula (II) with the amino compound of Formula (III) is performed at a pH range of 8 to 9.

15. The process as claimed in claim 1, wherein reacting the ketoamide of Formula (II) with the amino compound of Formula (III) is performed at a temperature ranging from 35° C. to 60° C.

16. The process as claimed in claim 4, further comprises adding a surface tension reducing agent or a phase transfer catalyst to the solution of amino compound of Formula (III) before adding the biocatalyst.

17. The process as claimed in claim 8, wherein the phase transfer catalyst is selected from tetrabutylammonium bromide (TBAB), tetrabutylammonium fluoride (TBAF), tetrabutylammonium hydroxide (TBAH), and triethylbenzylammonium chloride (TEBA).

18. The process as claimed in claim 4, wherein the buffer is triethanolamine.

19. The process as claimed in claim 12, wherein the solvent is isopropyl alcohol.

20. The process as claimed in claim 12, wherein the acid is selected from concentrated HCl, orthophosphoric acid, maleic acid, and fumaric acid.
